(19) United States
(12) Patent Application Publication
(10) Pub. No.: US 2002/0102729 A1
(43) Pub. Date: Aug. 1, 2002

(54) FORMULATIONS FOR ELECTROPORATION

(76) Inventors: FIONA MACLAUGHLIN, BELFAST (GB); SHULIN LI, THE WOODLANDS, TX (US); YUHUA LI, THE WOODLANDS, TX (US); KAREL PETRAK, SPRING, TX (US)

Correspondence Address: LYON & LYON LLP/ VALENTIS INC., 633 WEST FIFTH STREET, SUITE 4700, LOS ANGELES, CA, 90071-2066, US

(21) Appl. No.: 09/322,602

(22) Filed: May 28, 1999

Related U.S. Application Data
(60) Provisional application No. 60/088,691, filed on Jun. 8, 1998.

(51) Int. Cl. C12N 15/63; C12N 15/82; C07H 21/02

(52) U.S. Cl. 435/455; 435/468; 514/44; 536/23.1

(57) ABSTRACT

A novel method is provided for delivering nucleic acid molecules to the cells of an organism by pulse voltage delivery. The method involves the combination of formulated nucleic acid molecules with devices for injecting the molecules by pulse voltage or an electrical field. Disclosed are compositions and methods for enhancing the administration to and uptake of nucleic acids in a mammal. The methods disclosed provide an increased transfection and/or gene delivery efficiency by enhancing the uptake of formulated nucleic acid molecules by applying an electrical field which destabilizes the cellular membrane thereby opening pores or passageways which allow extracellular material to be introduced to the cell. Also disclosed are examples which demonstrate that the combination of formulated nucleic acid molecules and pulse voltage injection methods results in immune responses which are superior to those obtained by conventional means of delivery. Methods for delivery, as well as methods for formulating nucleic acid molecules with various compounds, such as cationic complexing agents, polymeric and non-polymeric formulations, protective, interactive, non-condensing systems are also disclosed.

The present invention relates to products and methods useful for delivering formulated nucleic acid molecules by pulse voltage delivery methods.

### BACKGROUND OF THE INVENTION

The following information is presented solely to assist the understanding of the reader, and none of the information is admitted to describe or constitute prior art to the claims of the present invention.

In the past, non-viral administration of nucleic acids in vivo has been pursued by a variety of methods. These include lipofectin/liposome fusion: Felgner et al., Proc. Natl. Acad. Sci., Volume 84, pp. 7413-7417 (1993); polylysine condensation with and without adenovirus enhancement: Curiel et al., Human Gene Therapy, Volume 3, pp. 147-154 (1992); and transferrin:transferrin receptor delivery of nucleic acid to cells: Wagner et al., Proc. Natl. Acad. Sci., Volume 87, pp. 3410-3414 (1990). The use of a specific composition consisting of polyacrylic acid has been disclosed in International Patent Publication No. WO 94/24983. Naked DNA has been administered as disclosed in International Patent Publication No. WO 90/11092.

Gene therapy has quickly become a major area of research in drug development. The key technological barrier to commercialization of gene therapy, however, is the need for practical and effective gene delivery methods. The primary problem of gene injection by conventional needle-syringe methods is that genetic material must be injected in large quantities into the target site because of the inefficiency of attempting to diffuse genetic material into the cells' nuclei and the need to overwhelm enzyme systems that immediately move to destroy the injected nucleic acid molecules. Since the introduction of the needle-syringe, therapeutic injection technology has progressed relatively slowly.

Injection by electroporation is a modern technique that involves the application of a pulsed electric field to create transient pores in the cellular membrane without causing permanent damage to the cell and thereby allows for the introduction of exogenous molecules. This technique has been used widely in research laboratories to create hybridomas and is now being applied to gene transfer approaches for therapy. By adjusting the electrical pulse generated by an electroporetic system, nucleic acid molecules can find their way through passageways or pores in the cell that are created during the procedure. U.S. Pat. No. 5,704,908 describes an electroporation apparatus for delivering molecules to cells at a selected location within a cavity in the body of a patient.

The use of electroporetic methods to deliver genes suspended in saline into rabbit and porcine arteries as models to treat coronary and peripheral vascular disease has been discussed at “The 3rd US-Japan Symposium on Drug Delivery” (D. B. Dev, J. J. Giordano and D. L. Brown., Maui, Hi., Dec. 17-22, 1995).

The ability to target and express the lacZ reporter gene suspended in saline to various depths of the dermis region in hairless mice has been described in the article “Depth-Targeted Efficient Gene delivery and Expression in the skin by Pulsed Electric Fields: An approach to Gene Therapy of Skin Aging and Other Diseases” (Zhang et al., Biochemical and Biophysical Research Communications 220, 633-636 (1996)).

A mammalian expression plasmid for the LacZ gene in saline has been injected into the internal carotid artery of rats whose brain tumors had been electroporated between two electrodes. The gene was said to be expressed in the tumor cells three days after plasmid injection and furthermore, lacZ activity was reported to be isolated only to the tissues and cells targeted (Nishi, et al., Cancer Research 56, 1050-1055, Mar. 1, 1996).

Despite these recent advances there remains need for additional and improved electroporation related injection products and methods.

### SUMMARY OF THE INVENTION

This invention features compositions and methods for enhancing the administration to and uptake of nucleic acids in an organism. An efficient strategy for enhancing pulse voltage delivery of nucleic acids in vivo is to protect the nucleic acid from degradation, thereby maintaining the administered nucleic acid at the target site in order to further increase its incorporation into the cells. The data presented herein demonstrates that the combination of formulated nucleic acid molecules and pulse voltage delivery methods is a more favorable method for nucleic acid delivery to specific tissues when compared with either pulse voltage delivery of non-formulated nucleic acids or non-pulse voltage injection of formulated nucleic acids.

The invention provides a method to deliver nucleic acid molecules formulated with an agent that facilitates transfection (preferably a PINC agent as described below) to an organism by using an apparatus configured and arranged to administer molecules by pulse voltage to the cells of an organism. Thus, the present invention allows for superior delivery of nucleic acid molecules into cells in vivo by the combination of a pulse voltage device and formulated nucleic acid molecules. Furthermore, the present invention also allows for treatment of diseases, vaccination, and treatment of muscle disorders and serum protein deficiencies.

In a first aspect, the present invention features a method for delivering a formulation of a nucleic acid molecule and a transfection facilitating agent to the cells of an organism by the use of a pulse voltage delivery device. Preferably, the pulse voltage injection device is configured and arranged to promote delivery of the formulation to and/or into the cells of the organism.

By “delivery” or “delivering” is meant transportation of nucleic acid molecules to desired cells or any cells. The nucleic acid molecules may be delivered to multiple cell lines, including the desired target. Delivery results in the nucleic acid molecules coming in contact with the cell surface, cell membrane, cell endosome, within the cell membrane, nucleus or within the nucleus, or any other desired area of the cell from which transfection can occur within a variety of cell lines which can include but are not limited to; tumor cells, epithelial cells, Langerhan cells, Langhans' cells, littoral cells, keratinocytes, dendritic cells, macrophage cells, kupffer cells, muscle cells, lymphocytes and lymph nodes. Preferably, the formulation is delivered to the cells by electroporation and the nucleic acid molecule component is not significantly sheared upon delivery, nor is cell viability directly effected by the pulse voltage delivery process.

The term “nucleic acid” as used herein refers to both RNA and DNA including: cDNA, genomic DNA, plasmid DNA or condensed nucleic acid, nucleic acid formulated with cationic lipids, nucleic acid formulated with peptides, antisense molecules, cationic substances, RNA or mRNA. In a preferred embodiment, the nucleic acid administered is plasmid DNA which includes a “vector”. The nucleic acid can be, but is not limited to, a plasmid DNA vector with a eukaryotic promoter which expresses a protein with potential therapeutic action, such as, for example; hGH, VEGF, EPO, IGF-1, IPO, Factor IX, IFN-α, IFN-β, IL-2, IL-12, or the like.

As used herein, the term a “plasmid” refers to a construct made up of genetic material (i.e., nucleic acids). It includes genetic elements arranged such that an inserted coding sequence can be transcribed in eukaryotic cells. Also, while the plasmid may include a sequence from a viral nucleic acid, such viral sequence preferably does not cause the incorporation of the plasmid into a viral particle, and the plasmid is therefore a non-viral vector. Preferably a plasmid is a closed circular DNA molecule.

The term “vector” as used herein refers to a construction comprised of genetic material designed to direct transformation of a targeted cell. A vector contains multiple genetic material, preferably contiguous fragments of DNA or RNA, positionally and sequentially oriented with other necessary elements such that the nucleic acid can be transcribed and when necessary translated in the transfected cells.

The term “transfection facilitating agent” as used herein refers to an agent that forms a complex with the nucleic acid. This molecular complex is associated with nucleic acid molecule in either a covalent or a non-covalent manner. The transfection facilitating agent should be capable of transporting nucleic acid molecules in a stable state and of releasing the bound nucleic acid molecules into the cellular interior. DNA extraction methods, methods of immunofluorescence, or well known reporter gene methods such as for example CAT, or LacZ containing plasmids, could be used in order to determine the transfection efficiency. The transfection facilitating agent should also be capable of being associated with nucleic acid molecules and lyophilized or freeze dried and either rehydrated prior to pulse voltage delivery.

In addition, the transfection facilitating agent may prevent lysosomal degradation of the nucleic acid molecules by endosomal lysis. Furthermore, the transfection facilitating agent may allow for efficient transport of the nucleic acid molecule through the cytoplasm of the cell to the nuclear membrane and into the nucleus and provide protection.

In a preferred embodiment transfection facilitating agents are non-condensing polymers, oils and surfactants. These may be suitable for use as compounds which prolong the localized bioavailability of a nucleic acid: polyvinylpyrrolidones; polyvinylalcohols; propylene glycols; polyethylene glycols; polyvinylacetates; poloxamers (Pluronics) (block copolymers of propylene oxide and ethylene oxide, relative amounts of the two subunits may vary in different poloxamers); poloxamines (Tetronics); ethylene vinyl acetates; celluloses, including salts of carboxymethylcelluloses, methylcelluloses, hydroxypropylcelluloses, hydroxypropylmethylcelluloses; salts of hyaluronates; salts of alginates; heteropolysaccharides (pectins); phosphatidylcholines (lecithins); miglyols; polylactic acid; polyhydroxybutyric acid. More preferably some of these compounds may be used as, and are considered protective, interactive, non-condensing compounds (PINC) and others as sustained release compounds, while some may be used in either manner under the respectively appropriate conditions.

In another embodiment cationic condensing agents such as cationic lipids, peptides, or lipopetides, or for example, dextrans, chitosans, dendrimers, polyethyleneimine (PEI), or polylysine, may associate with the nucleic acid molecule and may facilitate transfection after pulse voltage delivery.

The PINC enhances the delivery of the nucleic acid molecule to mammalian cells in vivo, and preferably the nucleic acid molecule includes a coding sequence for a gene product to be expressed in the cell. In many cases, the relevant gene product is a polypeptide or protein. Preferably the PINC is used under conditions so that the PINC does not form a gel, or so that no gel form is present at the time of administration at about 30-40° C. Thus, in these compositions, the PINC is present at a concentration of 30% (w/v) or less. In certain preferred embodiments, the PINC concentration is still less, for example, 20% or less, 10% or less, 5% or less, or 1% or less. Thus, these compositions differ in compound concentration and functional effect from uses of these or similar compounds in which the compounds are used at higher concentrations, for example in the ethylene glycol mediated transfection of plant protoplasts, or the formation of gels for drug or nucleic acid delivery. In general, the PINCs are not in gel form in the conditions in which they are used as PINCs, though certain of the compounds may form gels under some conditions.

In connection with the protective, interactive, non-condensing compounds for these compositions, the term “non-condensing” means that an associated nucleic acid is not condensed or collapsed by the interaction with the PINC at the concentrations used in the compositions. Thus, the PINCs differ in type and/or concentration from such condensing polymers. Examples of commonly used condensing polymers include polylysine, and cascade polymers (spherical polycations).

The term “protects” or “protective” or “protected” as used herein refers to an effect of the interaction between such a compound and a nucleic acid such that the rate of degradation of the nucleic acid is decreased in a particular environment, thereby prolonging the localized bioavailability of the nucleic acid molecule. Such degradation may be due to a variety of different factors, which specifically include the enzymatic action of a nuclease. The protective action may be provided in different ways, for example, by exclusion of the nuclease molecules or by exclusion of water.

The term “interactive” as used herein refers to the interaction between PINC's and nucleic acid molecules and/or cell wall components. Preferably, PINC polymers are capable of directly interacting with moieties of nucleic acid molecules and/or cell wall components. These interactions can facilitate transfection by, for example, helping associate the nucleic acid molecule-PINC complex closely with the cell wall as a result of biochemical interactions between the PINC and the cell wall and thereby mediate transfection. These interactions may also provide protection from nucleases by closely associating with the nucleic acid molecule.

Also in connection with such compounds and an associated nucleic acid molecule, the term “enhances the delivery” means that at least in conditions such that the amounts of PINC and nucleic acid is optimized, a greater biological effect is obtained than with the delivery of nucleic acid in saline. Thus, in cases where the expression of a gene product encoded by the nucleic acid is desired, the level of expression obtained with the PINC:nucleic acid composition is greater than the expression obtained with the same quantity of nucleic acid in saline for delivery by a method appropriate for the particular PINC/coding sequence combination.

In preferred embodiments of the above compositions, the PINC is polyvinyl pyrrolidone (PVP), polyvinyl alcohol (PVA), a PVP-PVA co-polymer, N-methyl-2-pyrrolidone (NM2P), ethylene glycol, or propylene glycol. In compositions in which a Poloxamer (Pluronics) is used, the nucleic acid is preferably not a viral vector, i.e., the nucleic acid is a non-viral vector.

In other preferred embodiments, the PINC is bound with a targeting ligand. Such targeting ligands can be of a variety of different types, including but not limited to galactosyl residues, fucosal residues, mannosyl residues, carntitine derivatives, monoclonal antibodies, polyclonal antibodies, peptide ligands, and DNA-binding proteins. The targeting ligands may bind with receptors on cells such as antigen-presenting cells, hepatocytes, myocytes, epithelial cells, endothelial cells, and cancer cells.

In connection with the association of a targeting ligand and a PINC, the term “bound with” means that the parts have an interaction with each other such that the physical association is thermodynamically favored, representing at least a local minimum in the free energy function for that association. Such interaction may involve covalent binding, or non-covalent interactions such as ionic, hydrogen bonding, van der Waals interactions, hydrophobic interactions, and combinations of such interactions.

While the targeting ligand may be of various types, in one embodiment the ligand is an antibody. Both monoclonal antibodies and polyclonal antibodies may be utilized.

The nucleic acid may also be present in various forms. Preferably the nucleic acid is not associated with a compounds(s) which alter the physical form, however, in other embodiments the nucleic acid is condensed (such as with a condensing polymer), formulated with cationic lipids, formulated with peptides, or formulated with cationic polymers.

In preferred embodiments, the protective, interactive non-condensing compound is polyvinyl pyrrolidone, and/or the plasmid is in a solution having between 0.5% and 50% PVP, more preferably about 5% PVP. The DNA preferably is at least about 80% supercoiled, more preferably at least about 90% supercoiled, and most preferably at least about 95% supercoiled.

In another aspect the invention features a composition containing a protective, interactive non-condensing compound and a plasmid containing an interferon alpha coding sequence.

In yet another aspect, the invention provides a PINC formulation of the invention as described above and a cationic lipid with a neutral co-lipid.

Preferably the cationic lipid is DOTMA and the neutral co-lipid is cholesterol (chol). DOTMA is 1,2-di-O-octadecenyl-3-trimethylammonium propane, which is described and discussed in Eppstein et al., U.S. Pat. No. 4,897,355, issued Jan. 30, 1990, which is incorporated herein by reference. However, other lipids and lipid combinations may be used in other embodiments. A variety of such lipids are described in Gao & Huang, 1995, Gene Therapy 2:710-722, which is hereby incorporated by reference.

As the charge ratio of the cationic lipid and the DNA is also a significant factor, in preferred embodiments the DNA and the cationic lipid are present is such amounts that the negative to positive charge ratio is about 1:3. While preferable, it is not necessary that the ratio be 1:3. Thus, preferably the charge ratio for the compositions is between about 1:1 and 1:10, more preferably between about 1:2 and 1:5.

The term “cationic lipid” refers to a lipid which has a net positive charge at physiological pH, and preferably carries no negative charges at such pH. An example of such a lipid is DOTMA. Similarly, “neutral co-lipid” refers to a lipid which has is usually uncharged at physiological pH. An example of such a lipid is cholesterol.

Thus, “negative to positive charge ratio” for the DNA and cationic lipid refers to the ratio between the net negative charges on the DNA compared to the net positive charges on the cationic lipid.

As the form of the DNA affects the expression efficiency, the DNA preferably is at least about 80% supercoiled, more preferably at least about 90% supercoiled, and most preferably at least about 95% supercoiled. The composition preferably includes an isotonic carbohydrate solution, such as an isotonic carbohydrate solution that consists essentially of about 10% lactose. In preferred embodiments, the composition the cationic lipid and the neutral co-lipid are prepared as a liposome having an extrusion size of about 800 nanometers. Preferably the liposomes are prepared to have an average diameter of between about 20 and 800 nm, more preferably between about 50 and 400 nm, still more preferably between about 75 and 200 nm, and most preferably about 100 nm. Microfluidization is the preferred method of preparation of the liposomes.

The compounds which protect the nucleic acid and/or prolong the localized bioavailability of a nucleic acid may achieve one or more of the following effects, due to their physical, chemical or rheological properties: (1) Protect nucleic acid, for example plasmid DNA, from nucleases due to steric, viscosity, or other effects such as shearing; (2) increase the area of contact between nucleic acid, such as plasmid DNA, through extracellular matrices and over cellular membranes, into which the nucleic acid is to be taken up; (3) concentrate nucleic acid, such as plasmid DNA, at cell surfaces due to water exclusion; (4) indirectly facilitate uptake of nucleic acid, such as plasmid DNA, by disrupting cellular membranes due to osmotic, hydrophobic or lytic effects; (5) indirectly facilitate uptake of nucleic acids by allowing diffusion of protected nucleic acid chains through tissue at the administration site; and (6) indirectly facilitate uptake of nucleic acid molecules through pore, holes, openings in the cells formed as a result of the electroporation process.

By “prolonging the localized bioavailability of a nucleic acid” is meant that a nucleic acid administered to an organism in a composition comprising a transfection facilitating agent will be available for uptake by cells for a longer period of time than if administered in a composition without such a compound, for example when administered in a saline solution. This increased availability of nucleic acid to cells could occur, for example, due to increased duration of contact between the composition containing the nucleic acid and a cell or due to protection of the nucleic acid from attack by nucleases. The compounds which prolong the localized bioavailability of a nucleic acid are suitable for internal administration.

By “suitable for internal administration” is meant that the compounds are suitable to be administered within the tissue of an organism, for example within a muscle or within a joint space, epidermally, intradermally or subcutaneously. Properties making a compound suitable for internal administration can include, for example, the absence of a high level of toxicity to the organism as a whole.

The term “pulse voltage device”, or “pulse voltage injection device” as used herein relates to an apparatus that is capable of causing or causes uptake of nucleic acid molecules into the cells of an organism by emitting a localized pulse of electricity to the cells, thereby causing the cell membrane to destabilize and result in the formation of passageways or pores in the cell membrane. It is understood that conventional devices of this type are calibrated to allow one of ordinary skill in the art to select and/or adjust the desired voltage amplitude and/or the duration of pulsed voltage and therefore it is expected that future devices that perform this function will also be calibrated in the same manner. The type of injection device is not considered a limiting aspect of the present invention. The primary importance of a pulse voltage device is, in fact, the capability of the device to deliver formulated nucleic acid molecules into the cells of an organism. The pulse voltage injection device can include, for example, an electroporetic apparatus as described in U.S. Pat. Nos. 5,439,440, 5,704,908 or U.S. Pat. No. 5,702,384 or as published in PCT WO 96/12520, PCT WO 96/12006, PCT WO 95/19805, and PCT WO 97/07826, all of which are incorporated herein by reference in their entirety.

The term “apparatus” as used herein relates to the set of components that upon combination allow the delivery of formulations of nucleic acid molecules and transfection facilitating agents into the cells of an organism by pulse voltage delivery methods.

Preferably, the apparatus is capable of being calibrated to allow selection of pulse voltage amplitude and duration.

The apparatus of the invention can be a combination of a syringe or syringes, various combinations of electrodes, devices which are useful for target selection by means such as optical fibers and video monitoring, and a generator for producing voltage pulses which can be calibrated for various voltage amplitudes, durations and cycles. The syringe can be of a variety of sizes and can be selected to inject formulations at different delivery depths such as to the skin of an organism such as a mammal, or through the skin.

The term “skin” refers to the outer covering of a mammal consisting of epidermal and dermal tissue and appendages such as sweat ducts and hair follicles. Skin can comprise the hair of a mammal in cases where the mammal has an epidermis which is covered by hair. In mammals which have enough hair to be considered fur or a pelt it is preferable to shave the hair, leaving primarily skin.

The term “organism” as used herein refers to common usage by one of ordinary skill in the art. The organism can include; micro-organisms ,such as yeast or bacteria, plants, birds, reptiles, fish or mammals. The organism can be a companion animal or a domestic animal. Preferably the organism is a mammal and is therefore any warm blooded organism. More preferably the mammal is a human.

The term “companion animal” as used herein refers to those animals traditionally treated as “pets” such as for example, dogs, cats, horses, birds, reptiles, mice, rabbits, hamsters, and the like.

The term “domestic animal” as used herein refers to those animals traditionally considered domesticated, where animals such as those considered “companion animals” are included along with animals such as, pigs, chickens, ducks, cows, goats, lambs, and the like.

In another embodiment the method results in an immune response, preferably a humoral immune response targeted for the protein product encoded by the nucleic acid molecule, such as an antibody response. In other situations the immune response preferably is a cytotoxic T-lymphocyte response.

The term “immune response” as used herein refers to the mammalian natural defense mechanism which can occur when foreign material is internalized. The immune response can be a global immune response involving the immune system components in their entirety. Preferably the immune response results from the protein product encoded by the formulated nucleic acid molecule. The immune response can be, but is not limited to; antibody production, T-cell proliferation /differentiation, activation of cytotoxic T-lymphocytes, and/or activation of natural killer cells. Preferably the immune response is a humoral immune response. However, as noted above, in other situations the immune response, preferably, is a cytotoxic T-lymphocyte response.

The term “humoral immune response” refers to the production of antibodies in response to internalized foreign material. Preferably the foreign material is the protein product encoded by a formulated nucleic acid molecule internalized by injection with a needle free device.

In a preferred embodiment the method results in enhanced transfection of cells as a result of a better method for gene delivery, when compared to pulse voltage or needle delivery of non-formulated (naked) nucleic acid. The enhanced transfection can be measured by transfection reporter methods commonly known in the art such as, for example, assays for CAT gene product activity, or LacZ gene product activity, and the like.

In another aspect the invention features a kit. The kit includes a container for providing a nucleic acid molecule formulated with a transfection facilitating agent and either, a pulse voltage device capable of being combined with the container for delivering nucleic acid molecules into the cells of an organism, and/or instructions explaining how to deliver the formulated nucleic acid molecules with a pulse voltage device.

Thus the “container” can include instructions furnished to allow one of ordinary skill in the art to make formulated nucleic acid molecules. The instructions will furnish steps to make the compounds used for formulating nucleic acid molecules. Additionally, the instructions will include methods for testing the formulated nucleic acid molecules that entail establishing if the formulated nucleic acid molecules are damaged upon injection after electroporation. The kit may also include notification of an FDA approved use and instructions.

The term “transfection” as used herein refers to the process of introducing DNA (e.g., formulated DNA expression vector) into a cell, thereby, allowing cellular transformation. Following entry into the cell, the transfected DNA may: (1) recombine with that of the host; (2) replicate independently as a plasmid or temperate phage; or (3) be maintained as an episome without replication prior to elimination.

As used herein, “transformation” relates to transient or permanent changes in the characteristics (expressed phenotype) of a cell induced by the uptake of a vector by that cell. Genetic material is introduced into a cell in a form where it expresses a specific gene product or alters the expression or effect of endogenous gene products.

Transformation of the cell may be associated with production of a variety of gene products including protein and RNA. These products may function as intracellular or extracellular structural elements, ligands, hormones, neurotransmitters, growth regulating factors, enzymes, chemotaxins, serum proteins, receptors, carriers for small molecular weight compounds, drugs, immunomodulators, oncogenes, cytokines, tumor suppressors, toxins, tumor antigens, antigens, antisense inhibitors, triple strand forming inhibitors, ribozymes, or as a ligand recognizing specific structural determinants on cellular structures for the purpose of modifying their activity. This list is only an example and is not meant to be limiting.

In another aspect, the invention features a method for making a kit. Preferably the method involves the step of combining a container for providing a nucleic acid formulated with a transfection facilitating agent with either, a pulse voltage device capable of being combined with the container, and/or instructions explaining how to deliver formulated nucleic acid molecules by pulse voltage.

In yet another aspect, the invention also features a method for treating a mammal that is suffering from a disorder conventionally treated by administering human growth hormone. The method requires administering a nucleic acid molecule encoding human growth hormone and formulated with a transfection facilitating agent into the cells of the mammal by use of a pulse voltage device.

In another aspect, the invention features a method for treating a mammal that is suffering from a cancer by administering a nucleic acid molecule encoding the appropriate cancer antigen. The method requires administering a nucleic acid molecule encoding a cancer antigen and formulated with a transfection facilitating agent into the cells of the mammal by use of a pulse voltage device.

In yet another aspect, the invention also features a method for treating a mammal that is suffering from an infectious disease by administering a nucleic acid molecule encoding an antigen for the infectious disease. The method requires administering a nucleic acid molecule encoding an antigen for the infectious disease and formulated with a transfection facilitating agent into the cells of the mammal by use of a pulse voltage device.

Administration as used herein refers to the route of introducing the formulated nucleic acid molecules of the invention into the body of cells or organisms. Administration includes the use of electroporetic methods as provided by a pulse voltage device to targeted areas of the mammalian body such as the muscle cells and the lymphatic cells in regions such as the lymph nodes.

Prior to administration, the nucleic acid molecules of the invention can be formulated with at least one transfection facilitating agent type of molecule. For example, the molecular complexes can be formulated with PINC's such as polyvinyl-pyrrolidone as described herein. Formulation techniques are provided herein by example.

The summary of the invention described above is not limiting and other features and advantages of the invention will be apparent from the following detailed description of the invention and from the claims.

### BRIEF DESCRIPTION OF THE DRAWINGS

FIG. 1 is a bar graph showing the transfection efficiency of plasmid injected by pulsed voltage delivery into cells of the gastrocnemius muscle of mice under PVP or PAcM formulated and non-formulated (saline) conditions.

FIG. 2 is a bar graph showing the transfection efficiency of plasmid delivered by pulsed voltage (1250-2000V/cm) delivery methods intratumorally. The figure shows the results of 2.5 ug of formulated (plasmid in 5% PVP and 0.9% NaCl) vs. non-formulated (plasmid in 0.9% NaCl) plasmid DNA containing a CAT reporter cassette after injection into renca tumors in experimental mice. The tumor cells were introduced to the experimental mice and allowed to grow to a reasonable size before injection. The results are given as CAT expression as determined by routine methods.

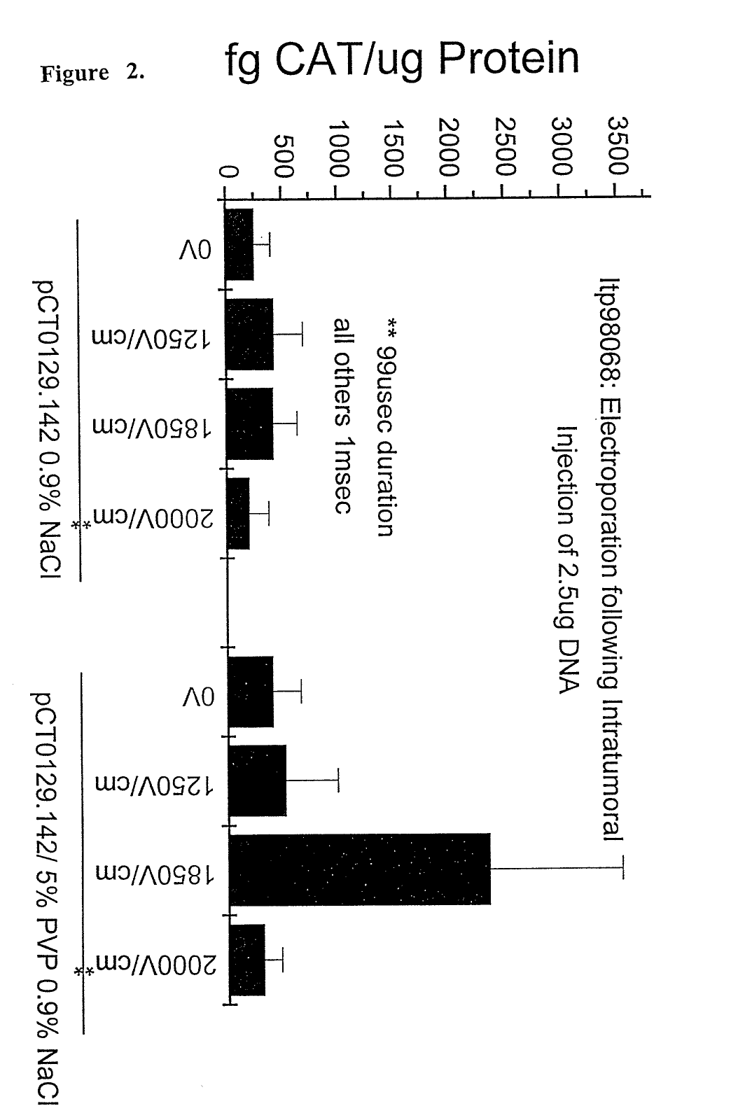

### DETAILED DESCRIPTION OF THE PREFERRED EMBODIMENTS

The delivery of formulations of nucleic acid molecules and transfection facilitating agents by the use of pulse voltage delivery device represents a novel approach to gene delivery. The present invention offers a nucleic acid delivery apparatus that provides, for example, an increased number of transfected cells, and also an increased immune response when compared to previous methods as a direct result of providing a more efficient method for transforming cell lines and, thereby increase the production of proteins which potentially trigger an immune response. The invention provides the advantage of allowing the uptake of formulated nucleic acid molecules by specifically targeted cells and cell lines, as well as uptake by multiple cell lines as desired. Injecting formulated nucleic acid molecules by pulse voltage delivery methods results in the formulated nucleic acid molecules gaining access to the cellular interior more directly through the destabilization of the cell wall and/or by the formation of pores as a result of the electroporetic process. Furthermore, in certain instances multiple cell lines can be targeted, thus allowing contact to many more cell types than in conventional needle injection. Thus, the present invention provides an enhanced delivery of nucleic acid molecules and also provides a more efficient gene delivery system which can be used to generate an immune response, modulate aspects of the cell cycle or cell physiology, or provide a method to achieve other gene delivery related therapeutic methods such as anti-tumor therapy.

Pulse voltage delivery of formulated nucleic acid molecules to an organism, depends on several factors which are discussed below, including transfection efficiency and the composition of the formulated nucleic acid molecule.

I. Preparation of Formulations

Formulations of nucleic acid molecules can be prepared as disclosed in Example 1. Substitute polymers are selected as determined by application. Generally, a weight/volume ratio is used as exemplified in both of the provided examples.

Delivery and expression of nucleic acids in many formulations, such as in saline, is limited due to degradation of the nucleic acids by cellular components of organisms, such as for instance nucleases. Thus, protection of the nucleic acids when delivered in vivo can greatly enhance the resulting expression, and thereby enhance a desired pharmacological or therapeutic effect. It was found that certain types of compounds which interact with a nucleic acid (e.g., DNA) in solution but do not condense the nucleic acid provide in vivo protection to the nucleic acid, and correspondingly enhance the expression of an encoded gene product. Some of these compounds have been discussed in U.S. Pat. No. 08/484,777, filed Jun. 7, 1998, International Patent Application No. PCT/US96/05679 filed Apr. 23, 1996 and U.S. patent application Ser. No. 60/045,295, filed May 2, 1997 all of which are incorporated herein by reference in their entirety including any drawings.

The use of delivery systems designed to interact with plasmids and protect plasmids from rapid extracellular nuclease degradation are described in, Mumper, R. J., et al., 1996, Pharm. Res. 13:701-709; Mumper, R. J., et al., 1997. Submitted to Gene Therapy. A characteristic of the PINC systems is that they are non-condensing systems that allow the plasmid to maintain flexibility and diffuse freely throughout the muscle while being protected from nuclease degradation. While the PINC systems are primarily discussed below, it will be understood that cationic lipid based systems and systems utilizing both PINCS and cationic lipids are also within the scope of the present invention.

A common structural component of the PINC systems is that they are amphiphilic molecules, having both a hydrophilic and a hydrophobic portion. The hydrophilic portion of the PINC is meant to interact with plasmids by hydrogen bonding (via hydrogen bond acceptor or donor groups), Van der Waals interactions, or/and by ionic interactions. For example, PVP and N-methyl-2-pyrrolidone (NM2P) are hydrogen bond acceptors while PVA and Propylene Glycol (PG) are hydrogen bond donors.

All four molecules have been reported to form complexes with various (poly)anionic molecules [Buhler V., BASF Aktiengescellschaft Feinchemie, Ludwigshafen, pp 39-42; Galaev Y, et al., J. Chrom. A. 684:45-54 (1994); Tarantino R, et al. J. Pharm. Sci. 83:1213-1216 (1994); Zia, H., et al., Pharm. Res. 8:502-504 (1991);]. The hydrophobic portion of the PINC systems is designed to result in a coating on the plasmid rendering its surface more hydrophobic. Kabanov et al. have described previously the use of cationic polyvinyl derivatives for plasmid condensation designed to increase plasmid hydrophobicity, protect plasmid from nuclease degradation, and increase its affinity for biological membranes [Kabanov, A. V., and Kabanov, V. A., 1995, Bioconj. Chem. 6:7-20; Kabanov, A. V., et al., 1991, Biopolymers 31:1437-1443; Yaroslavov, A. A., et al., 1996, FEBS Letters 384:177-180].

A substantial protective effect is observed; up to at least a one log enhancement of gene expression in rat muscle over plasmid formulated in saline has been demonstrated with these exemplary PINC systems. We have also found that the expression of reporter genes in muscle using plasmids complexed with the PINC systems was more reproducible than when the plasmid was formulated in saline. For example, the coefficient of variation for reporter gene expression in muscle using plasmid formulated in saline was 96±35% (n=20 studies; 8-12 muscles/study) whereas with coefficient of variation with plasmids complexed with PINC systems was 40±19% (n=30 studies; 8-12 muscles/study). The high coefficient of variation for reporter gene expression with plasmid formulated in saline has been described previously [Davis, H. L., et al., 1993, Hum. Gene Ther. 4:151-9]. In addition, in contrast with the results for DNA:saline, there was no significant difference in gene expression in muscle when plasmid with different topologies were complexed with polyvinyl pyrrolidone (PVP). This suggests that PVP is able to protect all forms of the plasmid from rapid nuclease degradation.

1. Summary of interactions between a PINC polymer (PVP) and plasmid.

We have demonstrated using molecular modeling that an exemplary PINC polymer, PVP, forms hydrogen bonds with the base pairs of a plasmid within its major groove and results in a hydrophobic surface on the plasmid due to the vinyl backbone of PVP. These interactions are supported by the modulation of plasmid zeta potential by PVP as well as by the inhibition of ethidium bromide intercalation into complexed plasmid. We have correlated apparent binding between PVP and plasmid to pH and salt concentration and have demonstrated the effect of these parameters on β-gal expression after intramuscular injection of plasmid/PVP complexes [Mumper, R. J., et al., 1997. Submitted to Gene Therapy]. A summary of the physico-chemical properties of plasmid/PVP complexes is listed in Table I below.

TABLE I
Summary of the Physico-Chemical Properties
of Plasmid/PVP Complexes

| Method | Result |
|---|---|
| Molecular modeling | Hydrogen bonding and hydrophobic |
| surface observed | plasmid |
| Fourier-transformed Infra-red | Hydrogen bonding demonstrated |
| DNase I challenge | Decreased rate of plasmid degradation |
| | in the presence of PVP |
| Microtitration Calorimetry | Positive heats of reaction indicative |
| | of an endothermic process |
| Potentiometric titration | One unit pH drop when plasmid |
| | and PVP are complexed |
| Dynamic Dialysis | Rate of diffusion of PVP reduced in |
| | the presence of plasmid |
| Zeta potential modulation | Surface charge of plasmid decreased by |
| | PVP |
| Ethidium bromide Intercalation | Ethidium bromide intercalation reduced |
| by plasmid/PVP complexation | |
| Osmotic pressure | Hyper-osmotic formulation (i.e., 340 |
| | mOsm/kg H₂O) |
| Luminescence Spectroscopy | Plasmid/PVP binding decreased in salt |
| | and/or at pH 7 |

2. Histology of expression in muscle.

Immunohistochemistry for β-gal using a slide scanning technology has revealed the uniform distribution of β-gal expression sites across the whole cross-sections of rat tibialis muscles. Very localized areas were stained positive for β-gal when CMV-β-gal plasmid was formulated in saline. β-gal positive cells were observed exclusively around the needle tract when plasmid was injected in saline. This is in agreement with previously published results [Wolff, J. A., et al., 1990, Science 247:1465-68; Davis, H. L., et al., 1993, Hum. Gene Ther. 4:151-9; Davis, H. L., et al., 1993, Hum. Gene Ther. 4:733-40].

In comparison, immunoreactivity for β-gal was observed in a wide area of muscle tissue after intramuscular injection of CMV-β-gal plasmid/PVP complex (1:17 w/w) in 150 mM NaCl. It appeared that the majority of positive muscle fibers were located at the edge of muscle bundles. Thus, staining for β-gal in rat muscle demonstrated that, using a plasmid/PVP complex, the number of muscle fibers stained positive for β-gal was approximately 8-fold greater than found using a saline formulation. Positively stained muscle fibers were also observed over a much larger area in the muscle tissue using the plasmid/PVP complex providing evidence that the injected plasmid was widely dispersed after intramuscular injection.

One conclusion is that the enhanced plasmid distribution and expression in rat skeletal muscle was a result of both protection from extracellular nuclease degradation due to complexation and hyper-osmotic effects of the plasmid/PVP complex. However, Dowty and Wolff et al. have demonstrated that osmolarity, up to twice physiologic osmolarity, did not significantly affect gene expression in muscle [Dowty, M. E., and Wolff, J. A. In: J. A. Wolff (Ed.), 1994, Gene Therapeutics: Methods and Applications of Direct Gene Transfer. Birkhauser, Boston, pp. 82-98]. This suggests that the enhanced expression of plasmid due to PVP complexation is most likely due to nuclease protection and less to osmotic effects. Further, the surface modification of plasmids by PVP (e.g., increased hydrophobicity and decreased negative surface charge) may also facilitate the uptake of plasmids by muscle cells.

3. Structure-activity relationship of PINC polymers.

A linear relationship between the structure of a series of co-polymers of vinyl pyrrolidone and vinyl acetate and the levels of gene expression in rat muscle has been found. Also, the substitution of some vinyl pyrrolidone monomers with vinyl acetate monomers in PVP results in a co-polymer with reduced ability to form hydrogen bonds with plasmids. The reduced interaction subsequently led to decreased levels of gene expression in rat muscle after intramuscular injection. The expression of β-gal decreased linearly (R=0.97) as the extent of vinyl pyrrolidone monomer (VPM) content in the co-polymers decreased.

These data demonstrate that pH and viscosity are not the most important parameters effecting delivery of plasmid to muscle cells since these values were equivalent for all complexes. These data suggest that enhanced binding of the PINC polymers to plasmid results in increased protection and bioavailability of plasmid in muscle.

4. Additional PINC systems.

The structure-activity relationship described above can be used to design novel co-polymers that will also have enhanced interaction with plasmids. It is expected that there is “an interactive window of opportunity” whereby enhanced binding affinity of the PINC systems will result in a further enhancement of gene expression after their intramuscular injection due to more extensive protection of plasmids from nuclease degradation. It is expected that there will be an optimal interaction beyond which either condensation of plasmids will occur or “triplex” type formation, either of which can result in decreased bioavailability in muscle and consequently reduced gene expression.

As indicated above, the PINC compounds are generally amphiphilic compounds having both a hydrophobic portion and a hydrophilic portion. In many cases the hydrophilic portion is provided by a polar group. It is recognized in the art that such polar groups can be provided by groups such as, but not limited to, pyrrolidone, alcohol, acetate, amine or heterocyclic groups such as those shown on pp. 2-73 and 2-74 of CRC Handbook of Chemistry and Physics (72nd Edition), David R. Lide, editor, including pyrroles, pyrazoles, imidazoles, triazoles, dithiols, oxazoles, (iso)thiazoles, oxadiazoles, oxatriazoles, diaoxazoles, oxathioles, pyrones, dioxins, pyridines, pyridazines, pyrimidines, pyrazines, piperazines, (iso)oxazines, indoles, indazoles, carpazoles, and purines and derivatives of these groups, hereby incorporated by reference.

Compounds also contain hydrophobic groups which, in the case of a polymer, are typically contained in the backbone of the molecule, but which may also be part of a non-polymeric molecule. Examples of such hydrophobic backbone groups include, but are not limited to, vinyls, ethyls, acrylates, acrylamides, esters, celluloses, amides, hydrides, ethers, carbonates, phosphazenes, sulfones, propylenes, and derivatives of these groups. The polarity characteristics of various groups are quite well known to those skilled in the art as illustrated, for example, by discussions of polarity in any introductory organic chemistry textbook.

The ability of such molecules to interact with nucleic acids is also understood by those skilled in the art, and can be predicted by the use of computer programs which model such intermolecular interactions. Alternatively or in addition to such modeling, effective compounds can readily be identified using one or more of such tests as 1) determination of inhibition of the rate of nuclease digestion, 2) alteration of the zeta potential of the DNA, which indicates coating of DNA, 3) or inhibition of the ability of intercalating agents, such as ethidium bromide to intercalate with DNA.

5. Targeting Ligands.

In addition to the nucleic acid/PINC complexes described above for delivery and expression of nucleic acid sequences, in particular embodiments it is also useful to provide a targeting ligand in order to preferentially obtain expression in particular tissues, cells, or cellular regions or compartments.

Such a targeted PINC complex includes a PINC system (monomeric or polymeric PINC compound) complexed to plasmid (or other nucleic acid molecule) . The PINC system is covalently or non-covalently attached to (bound to) a targeting ligand (TL) which binds to receptors having an affinity for the ligand. Such receptors may be on the surface or within compartments of a cell. Such targeting provides enhanced uptake or intracellular trafficking of the nucleic acid.

The targeting ligand may include, but is not limited to, galactosyl residues, fucosal residues, mannosyl residues, carnitine derivatives, monoclonal antibodies, polyclonal antibodies, peptide ligands, and DNA-binding proteins. Examples of cells which may usefully be targeted include, but are not limited to, antigen-presenting cells, hepatocytes, myocytes, epithelial cells, endothelial cells, and cancer cells.

Formation of such a targeted complex is illustrated by the following example of covalently attached targeting ligand (TL) to PINC system:

TL-PINC+Plasmid→TL-PINC::::::Plasmid

Formation of such a targeted complex is also illustrated by the following example of non-covalently attached targeting ligand (TL) to PINC system.

TL::::::PINC+Plasmid→TL::::::PINC::::::Plasmid

or alternatively,

PINC+Plasmid→PINC:::::::Plasmid+TL→TL::::::PINC:::::::Plasmid

In these examples :::::::: is non-covalent interaction such as ionic, hydrogen-bonding, Van der Waals interaction, hydrophobic interaction, or combinations of such interactions.

A targeting method for cytotoxic agents is described in Subramanian et al., International Application No. PCT/US96/08852, International Publication No. WO 96/39124, hereby incorporated by reference. This application describes the use of polymer affinity systems for targeting cytotoxic materials using a two-step targeting method involving zip polymers, i.e., pairs of interacting polymers. An antibody attached to one of the interacting polymers binds to a cellular target. That polymer then acts as a target for a second polymer attached to a cytotoxic agent. As referenced in Subramanian et al., other two-step (or multi-step) systems for delivery of toxic agents are also described.

In another aspect, nucleic acid coding sequences can be delivered and expressed using a two-step targeting approach involving a non-natural target for a PINC system or PINC-targeting ligand complex. Thus, for example, a PINC-plasmid complex can target a binding pair member which is itself attached to a ligand which binds to a cellular target (e.g., a MAB). Binding pairs for certain of the compounds identified herein as PINC compounds as identified in Subramanian et al. Alternatively, the PINC can be complexed to a tareting ligand, such as an antibody. That antibody can be targeted to a non-natural target which binds to, for example, a second antibody.

II. Administration

Administration as used herein refers to the route of introduction of a plasmid or carrier of DNA into the body. Administration can be directly to a target tissue or by targeted delivery to the target tissue after systemic administration. In particular, the present invention can be used for treating conditions by administration of the formulation to the body in order to establish controlled expression of any specific nucleic acid sequence within tissues at certain levels that are useful for gene therapy.

The preferred means for administration of vector (plasmid) and use of formulations for delivery are described above. The preferred embodiments are by pulse voltage delivery to cells in combination with needle or needle free injection, or by direct applied pulse voltage wherein the electroporation device's electrodes are pressed directly against the targeted tissue or cells, such as for example epidermal cells, and the vector is applied topically before or after pulse application and delivered through and or to the cells.

The route of administration of any selected vector construct will depend on the particular use for the expression vectors. In general, a specific formulation for each vector construct used will focus on vector delivery with regard to the particular targeted tissue, the pulse voltage delivery parameters, followed by demonstration of efficacy. Delivery studies will include uptake assays to evaluate cellular uptake of the vectors and expression of the DNA of choice. Such assays will also determine the localization of the target DNA after uptake, and establishing the requirements for maintenance of steady-state concentrations of expressed protein. Efficacy and cytotoxicity can then be tested. Toxicity will not only include cell viability but also cell function.

Muscle cells have the unique ability to take up DNA from the extracellular space after simple injection of DNA particles as a solution, suspension, or colloid into the muscle. Expression of DNA by this method can be sustained for several months.

Delivery of formulated DNA vectors involves incorporating DNA into macromolecular complexes that undergo endocytosis by the target cell. Such complexes may include lipids, proteins, carbohydrates, synthetic organic compounds, or inorganic compounds. Preferably, the complex includes DNA, a cationic lipid, and a neutral lipid in particular proportions. The characteristics of the complex formed with the vector (size, charge, surface characteristics, composition) determines the bioavailability of the vector within the body. Other elements of the formulation function as ligand which interact with specific receptors on the surface or interior of the cell. Other elements of the formulation function to enhance entry into the cell, release from the endosome, and entry into the nucleus.

Delivery can also be through use of DNA transporters. DNA transporters refers to molecules which bind to DNA vectors and are capable of being taken up by epidermal cells. DNA transporters contain a molecular complex capable of noncovalently binding to DNA and efficiently transporting the DNA through the cell membrane. It is preferable that the transporter also transport the DNA through the nuclear membrane. See, e.g., the following applications all of which (including drawings) are hereby incorporated by reference herein: (1) Woo et al., U.S. Ser. No. 07/855,389, entitled “A DNA Transporter System and Method of Use”, filed Mar. 20, 1992, now abandoned; (2) Woo et al., PCT/US93/02725, International Publ. WO93/18759, entitled “A DNA Transporter System and Method of Use”, (designating the U.S. and other countries) filed Mar. 19, 1993; (3) continuation-in-part application by Woo et al., entitled “Nucleic Acid Transporter Systems and Methods of Use”, filed Dec. 14, 1993, U.S. Ser. No. 08/167,641; (4) Szoka et al. , U.S. Ser. No. 07/913,669, entitled “Self-Assembling Polynucleotide Delivery System”, filed Jul. 14, 1992 and (5) Szoka et al., PCT/US93/03406, International Publ. WO93/19768 entitled “Self-Assembling Polynucleotide Delivery System”, (designating the U.S. and other countries) filed Apr. 5, 1993.

A DNA transporter system can consist of particles containing several elements that are independently and non-covalently bound to DNA. Each element consists of a ligand which recognizes specific receptors or other functional groups such as a protein complexed with a cationic group that binds to DNA. Examples of cations which may be used are spermine, spermine derivatives, histone, cationic peptides and/or polylysine.

One element is capable of binding both to the DNA vector and to a cell surface receptor on the target cell. Examples of such elements are organic compounds which interact with the asialoglycoprotein receptor, the folate receptor, the mannose-6-phosphate receptor, or the carnitine receptor.

A second element is capable of binding both to the DNA vector and to a receptor on the nuclear membrane. The nuclear ligand is capable of recognizing and transporting a transporter system through a nuclear membrane. An example of such ligand is the nuclear targeting sequence from SV40 large T antigen or histone.

A third element is capable of binding to both the DNA vector and to elements which induce episomal lysis. Examples include inactivated virus particles such as adenovirus, peptides related to influenza virus hemagglutinin, or the GALA peptide described in the Szoka patent cited above.

Administration may also involve lipids as described in preferred embodiments above. The lipids may form liposomes which are hollow spherical vesicles composed of lipids arranged in unilamellar, bilamellar, or multilamellar fashion and an internal aqueous space for entrapping water soluble compounds, such as DNA, ranging in size from 0.05 to several microns in diameter. Lipids may be useful without forming liposomes. Specific examples include the use of cationic lipids and complexes containing DOPE which interact with DNA and with the membrane of the target cell to facilitate entry of DNA into the cell.

The chosen method of delivery should result in expression of the gene product encoded within the nucleic acid cassette at levels which exert an appropriate biological effect. The rate of expression will depend upon the disease, the pharmacokinetics of the vector and gene product, and the route of administration, but should be in the range 0.001-100 mg/kg of body weight/day, and preferably 0.01-10 mg/kg of body weight/day. This level is readily determinable by standard methods. It could be more or less depending on the optimal dosing. The duration of treatment will extend through the course of the disease symptoms, possibly continuously. The number of doses will depend upon the disease, delivery vehicle, and efficacy data from clinical trials.

III. DNA Injection Variables

The level of gene delivery and expression or the intensity of an immune response achieved with the present invention can be optimized by altering the following variables. The variables are: the formulation (composition, plasmid topology), the technique and protocol for injection (area of injection, duration and amplitude of voltage, electrode gap, number of pulses emitted, type of needle arrangement, pre-injection-pulsed or post-injection-pulsed cells, state of muscle, state of the tumor), and, the pretreatment of the muscle with myotoxic agents. An immune response can be measured by, but is not limited to, the amount of antibodies produced for a protein encoded and expressed by the injected nucleic acid molecule.

Other injection variables that can be used to significantly affect the levels of proteins, antibodies and/or cytotoxic T-lymphocytes produced in response to the protein encoded by the formulated nucleic acid molecule provided by the pulse voltage injection method of the present invention are the state of the muscle being injected and injection technique. Examples of the variables include muscle stimulation, muscle contraction, muscle massage, delivery angle, and apparatus manipulation. Massaging the muscle may force plasmid out of the muscle either directly or via lymphatic drainage. By altering the depth of penetration and/or the angle at which the pulse voltage device is placed in relation to muscle fibers the present invention improves the plasmid distribution throughout the injection area which subsequently increases the antibody response to the protein which is encoded and expressed by the plasmid.

IV. Nucleic Acid Based Therapy

The present invention can be used to deliver nucleic acid vaccines in a more efficient manner than is conventionally done at the present time. Nucleic acid vaccines, or the use of plasmid encoding antigens or therapeutic molecules such as Human Growth Hormone, has become an area of intensive research and development in the last half decade. Comprehensive reviews on nucleic acid based vaccines have been published [M. A. Liu, et al.(Eds.), 1995, DNA Vaccines: A new era in vaccinology, Vol. 772, Ann. NY. Acad. Sci., New York; Kumar, V., and Sercarz, E., 1996, Nat. Med. 2:857-859; Ulmer, J. B., et al., (Eds.) Current Opinion in Immunology; 8:531-536. Vol. 772, Ann. NY. Acad. Sci., New York]. Protective immunity in an animal model using plasmid encoding a viral protein was first observed in 1993 by Ulmer et al. [Ulmer, J. B., et al., 1993, Science 259:1745-1749]. Since then, several studies have demonstrated protective immunity for several disease targets and human clinical trials have been started.

Many disease targets have been investigated. Examples include antigens of Borrelia burgdorferi, the tick-borne infectious agent for Lyme disease (Luke et al., J. Infect. Dis. 175:91-97, 1997), human immunodeficiency virus-1, (Letvin et al., Proc. Nat. Acad. Sci. USA 94:9378-9383, 1997), B cell lymphoma (Syrengelas et al., Nature Medicine. 2:1038-41, 1996), Herpes simplex virus (Bourne et al., J. Infectious dis. 173:800-807, 1996), hepatitis C virus (Tedeschi et al., Hepatology 25:459-462, 1997), rabies virus (Xiang et al., virology, 209:569-579, 1995), Mycobacterium tuberculosis (Lowrie in Genetic Vaccines and Immunotherapeutic Strategies C A Thibeault, ed. Intl Bus Comm, Inc., southborough, MA 01772 pp. 87-122, 1996), and Plasmodium falciparum (Hoffman et al., Vaccine 15:842-845, 1997). Additionally, nucleic acid based treatment for reducing tumor-cell immunogenicity, growth, and proliferation is indicative of gene therapy for diseases such as tumorigenic brain cancer (Fakhrai et al., Proc. Natl. Acad. Sci., 93:2909-2914, 1996).

An important goal of gene therapy is to affect the uptake of nucleic acid by cells, thereby causing an immune response to the protein encoded by the injected nucleic acid. Uptake of nucleic acid by cells is dependent on a number of factors, one of which is the length of time during which a nucleic acid is in proximity to a cellular surface. The present invention provides formulations which increase the length of time during which a nucleic acid is in proximity to a cellular surface, and penetrate the cell thereby delivering nucleic acid molecules into the cell.

Nucleic acid based vaccines are an attractive alternative vaccination strategy to subunit vaccines, purified viral protein vaccines, or viral vector vaccines. Each of the traditional approaches has limitations that are overcome if the antigen(s) is expressed directly in cells of the body. Furthermore, these traditional vaccines are only protective in a strain-specific fashion. Thus, it is very difficult, and even impossible using traditional vaccine approaches to obtain long lasting immunity to viruses that have several sera types or viruses that are prone to mutation.

Nucleic acid based vaccines offer the potential to produce long lasting immunity against viral epitopes that are highly conserved, such as with the nucleoprotein of viruses. Injecting plasmids encoding specific proteins by the present invention results in increased immune responses, as measured by antibody production. Thus, the present invention includes new methods of providing nucleic acid vaccines by delivering a formulated nucleic acid molecule with a pulse voltage device as described herein.

The efficacy of nucleic acid vaccines is enhanced by one of at least three methods: (1) the use of delivery systems to increase the stability and distribution of plasmid within the muscle, (2) by the expression (or delivery) of molecules to stimulate antigen presentation/transfer, or (3) by the use of adjuvants that may modulate the immune response.

V. Polymeric and Non-polymeric Formulations for Plasmid Delivery

The present invention provides polymeric and non-polymeric formulations which address problems associated with injection of nucleic acids suspended in saline. Unformulated (naked nucleic acid molecules) plasmids suspended in saline have poor bioavailability in muscle due to rapid degradation of plasmid by extracellular nucleases. One possible approach to overcome the poor bioavailability is to protect plasmid from rapid nuclease degradation by for example condensing the plasmid with commonly used cationic complexing agents. However, due to the physiology of the muscle, the use of rigid condensed particles containing plasmid for efficient transfection of a larger number of muscle cells has not been successful to date. Cationic lipid and polylysine plasmid complexes do not cross the external lamina to gain access to the caveolae and T tubules [Wolff, J. A., et al., 1992, J. Cell. Sci. 103:1249-1259].

Thus, the strategy identified for increasing the bioavailability of plasmid in muscle was to: protect plasmid from rapid extracellular nuclease degradation, disperse and retain intact plasmid in the muscle and/or tumor, and facilitate the uptake of plasmid by muscle and/or tumor cells. A specific method of accomplishing this, which preferably is used in conjunction with pulse voltage delivery, is the use of protective, interactive, non-condensing systems (PINC).

VI. Diseases and Conditions for Intramuscular Plasmid Delivery

The present invention described herein can be utilized for the delivery and expression of many different coding sequences. In particular, the demonstrated effectiveness for the PINC systems (PCT Application No. PCT/US96/05679) for delivery to muscle indicate that such formulations are effective for delivery of a large variety of coding sequences to muscle by pulse voltage injection. As transforming muscle and other cells has been shown to be effective, in an additional aspect of the invention tumor cells are also targeted for pulse voltage injection. Hence, the present invention provides methods for treating cancerous conditions associated with the formation of tumors or aggregated cell colonies such as those found in conditions such as skin cancer and the like. Specific suggestions for delivery of coding sequences to muscle cells with the pulse voltage device of the present invention include those summarized in Table 2 below.

TABLE 2
Applications for Plasmid-Based Gene Therapy by Intramuscular Injection

References are numbered as they are cited in U.S.
application No. PCT/US96/05679, which has been
incorporated by reference in its entirety.

Muscle and nerve disorders

| | |
|---|---|
| Duchenne's muscular dystrophy [6], Miller 1995 [7] | Acsadi 1991 [5], Karpati 1993 |
| Myotrophic disorders (IGF-I) 1997 [9] | Coleman 1997 [8], Alila |
| Neurotrophic disorders (IGF-I) [10] | Alila 1997 [9], Rabinovsky 1997 |
| Secretion of expressed protein into the systemic circulation | |
| Hemophilias A and B 1994 [12], Miller 1994 [13] | Anwer 1996 [11], Kuwahara-Rundell |
| Erythropoietin-responsive | Tripathy 1996 [14] |
| Pituitary dwarfism | Anwer 1996 [11], Dahler 1994 [15] |
| α1-Antitrypsin deficiency | Levy 1996 [16] |
| Autoimmune and Inflammatory diseases | Raz 1993 [17] |
| Hypercholesterolema | Fazio 1994 [18] |
| Hypotension | Ma 1995 [19] |
| Hypertension | Xiong 1995 [20] |
| Nucleic acid vaccines | |
| Herpes Simplex Virus [22], McClements 1996 [23], Kriesel 1996 [24] | Manickan 1995 [21], Ghiasi 1995 |
| Hepatitis B Virus [26], Davis 1996 [27] | Davis 1993 [25], Davis 1994 |
| Influenza Virus [29], Ulmer 1994 [30] | Donnelly 1995 [28], Ulmer 1993 |
| Tuberculosis | Lowrie 1994 [31], Tascon, 1996 [32] |
| Human Immunodeficiency Virus [34], Wang 1993 [35] | Shiver 1995 [33], Coney 1994 |
| Cancer | Raz 1993 [17], Russell 1994 [36] |
| Maleria [38] | Hoffman 1995 [37], Sedegah 1994 |
| Hepatitis C virus [40] | Major 1995 [39], Lagging 1995 |
| Flavivirus | Phillpotts 1996 [41] |
| Cytomegalovirus | Pande 1995 [42] |
| Salmonella typhi | Lopez-Macias 1995 [43] |
| Mycoplasma pulmonis | Lai 1995 [44] |
| Rabies virus | Xiang 1995 [45] |

Diseases to be Treated

The condition or disease preferably is a cancer, such as epithelial glandular cancer, including adenoma and adenocarcinoma; squamous and transitional cancer, including polyp, papilloma, squamous cell and transitional cell carcinoma; connective tissue cancer, including tissue type positive, sarcoma and other (oma's); hematopoietic and lymphoreticular cancer, including lymphoma, leukemia and Hodgkin's disease; neural tissue cancer, including neuroma, sarcoma, neurofibroma and blastoma; mixed tissues of origin cancer, including teratoma and teratocarcinoma. Other cancerous conditions that are applicable to treatment include cancer of any of the following: adrenal gland, anus, bile duct, bladder, brain tumors: adult, breast, cancer of an unknown primary site, carcinoids of the gastrointestinal tract, cervix, childhood cancers, colon and rectum, esophagus, gall bladder, head and neck, islet cell and other pancreatic carcinomas, Kaposi's sarcoma, kidney, leukemia, liver, lung: non-small cell, lung: small cell, lymphoma: AIDS-associated, lymphoma: Hodgkin's disease, Lymphomas: non-Hodgkin's disease, melanoma, mesothelioma, metastatic cancer, multiple myeloma, ovary, ovarian germ cell tumors, pancreas, parathyroid, penis, pituitary, prostate, sarcomas of bone and soft tissue, skin, small intestine, stomach, testis, thymus, thyroid, trophoblastic disease, uterus: endometrial carcinoma, uterus: uterine sarcomas, vagina, or vulva. The composition preferably is administered by pulsed voltage delivery and may require, as needed, exposure of the tissue to be treated by surgical means as determined by a certified professional.

### EXAMPLES

The following examples are offered by way of illustration and are not intended to limit the scope of the invention in any manner. One of ordinary skill in the art would recognize that the various molecules and/or amounts disclosed in the examples could be adjusted or substituted by larger amounts (for larger scaled experiments) or by inclusion of a different Transfection Facilitating Agent. It would also be recognized that the delivery targets and/or amounts delivered in the examples could be adjusted or substituted by selecting different muscles for injection, injection into tumors or nodes, or increasing or decreasing the duration of pulse time or alternating the pulse application from pre-injection to post-injection.

### Example 1

Demonstration of Transfection Facilitating Agent-Plasmid DNA Complex Formation

Preparation of PVP Formulated Nucleic Acid Molecules

Concentrated pDNA stock solutions were made by lyophilizing and rehydrating PDNA with water to a final pDNA concentration of 2-5 mg/ml. Formulations were made by aliquoting appropriate volumes of sterile stock solutions of PDNA, 5M NaCL, and polymer to obtain a final pDNA concentration in an isotonic polymer solution. Stock solutions were added in the following order: water, plasmid, polymer, and 5M NaCl. The plasmid and polymers were allowed to incubate at room temperature for 15 minutes prior to adding salt or lactose for ionicity adjustments. Likewise, Na-citrate buffers in 0.9% NaCl were added after incubating the plasmid and polymers for 15 minutes at room temperature. The osmotic pressure of selected formulations was measured (n=3) using a Fiske One-Ten Micro-Sample Osmometer. The pH of all formulations was measured using an Accumet Model 15 pH Meter and the viscosity of all formulations was measured using a Programmable Rheometer Model DV-III.

Dynamic dialysis was used with various interactive polymer formulations to measure binding between PVP and plasmid DNA. One ml of formulations and corresponding controls were place in prewashed dialysis sacs. The dialysis sacs were closed and suspended in stirred saline solutions (100 ml) at 25° C. One ml aliquots were taken from the acceptor compartment over time and replaced with fresh media. The concentration of PVP in the diffused samples collected over time was measured spectroscopically at 220 nm.

In all cases, the rate of PVP diffusion through the dialysis membrane was decreased in the presence of plasmid DNA, indicating complex formation between PVP and plasmid DNA. The reduction in the diffusion rate for PVP in the presence of plasmid DNA was directly proportional to the initial amount of PVP in the dialysis sac. It was also determined that the sac volume remained constant during the duration of the experiment and that adherence of PVP to the membrane was negligible.

### Example 2

Comparison of the Effect of Electroporation on Expression of ‘Naked’ Plasmid Vs. PVP or PAcM Formulated Plasmid

Design

Animals: 51 CD-1 mice (30 g) from Harlan-Sprague Dawley Inc. The animals were housed in microisolators at five mice per isolator in the laboratory animal resource vivarium and maintained at a 12/12 hr day/night cycle, at room temperature (72° C.)

Anesthesia: Combination anesthetic methods were used as follows; a mixture of Ketamine (74.0 mg/ml), Xylazine (3.7 mg/ml) and Acepromazine (0.73 mg/ml) was administered intraperitoneal at a dosage of 1.8-2.0 ml/kg.

Formulations: Plasmid containing Luciferase reporter cassette at concentration of 5.2 mg/ml was formulated as follows, and then injected intramuscularly into each leg—50 μl (two 25 μl injections) in each gastrocnemius muscle.

| | | | Pulse | |
|---|---|---|---|---|
| Formulation | Plasmid conc. | Dose | 500 V | 800 |
| Plasmid/.9% saline | 4 mg/ml | 200μg n = 10 | n = 10 | |
| ″/PVP 5% | ″ | ″ | ″ | ″ |
| ″/PAcM | ″ | ″ | ″ | ″ |
| naive | n/a | | n = 4 | n = 4 |

Pulse voltage delivery at 2 minutes after needle administration of formulation.

The gastrocnemius muscles were harvested after 2 days, collected on dry ice, lyophilized, and stored at −80° C.

Results

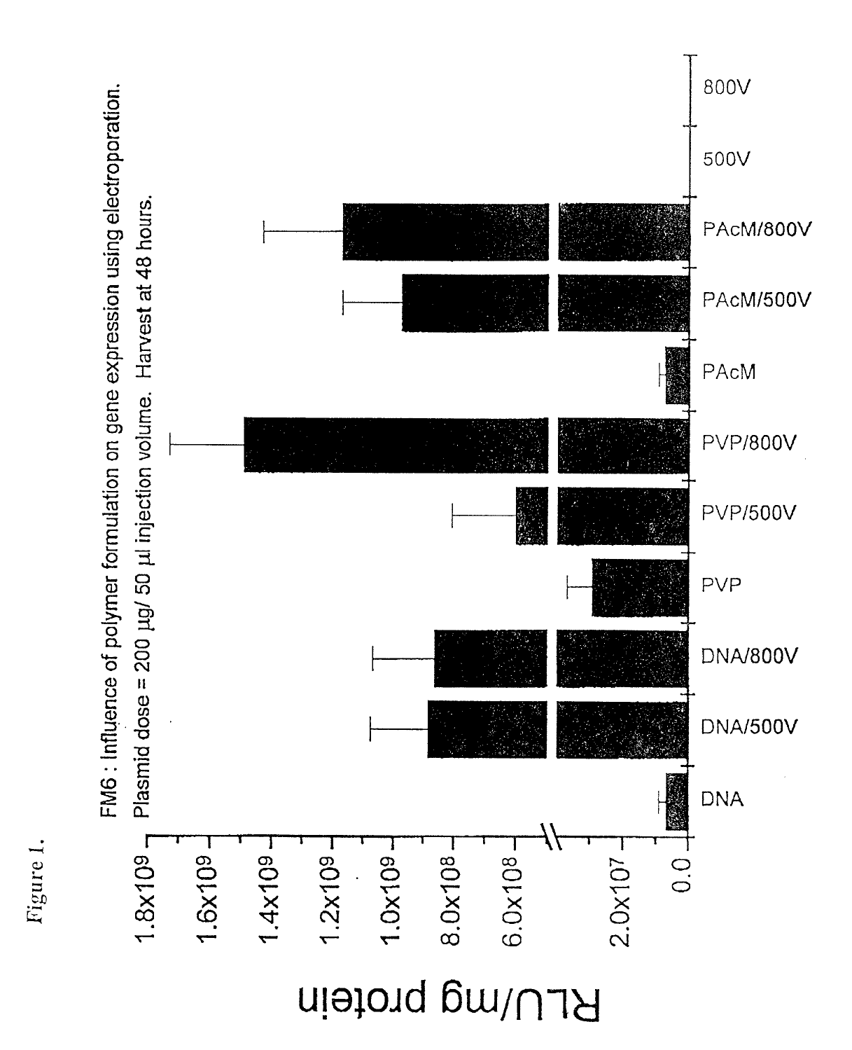

Pulse voltage delivery of both the PVP and PACM formulated plasmid resulted in an observed a higher level of measured fluorescence when compared to DNA formulated in Saline (FIG. 1).

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Those references not previously incorporated herein by reference, including both patent and non-patent references, are expressly incorporated herein by reference for all purposes. Other embodiments are within the following claims.

What is claimed is:

1. A method for delivering a nucleic acid molecule to an organism comprising the step of providing a formulation comprising said nucleic acid molecule and a transfection facilitating agent to the cells of said organism by use of a device configured and arranged to cause pulse voltage delivery of said formulation.

2. The method of claim 1, wherein said nucleic acid molecule is DNA.

3. The method of claim 1, wherein said nucleic acid molecule is one or more plasmids with a eukaryotic promoter which expresses one or more therapeutic molecules.

4. The method of claim 3, wherein said therapeutic molecule is for human growth hormone.

5. The method of claim 1, wherein said nucleic acid molecule is RNA.

6. The method of claim 1, wherein said transfection facilitating agent is a protective, interactive and non-condensing compound.

7. The method of claim 1, wherein said transfection facilitating agent is selected from the group consisting of: one or more polyvinyl-pyrrolidones, one or more cationic lipids, one or more cationic lipids with neutral co-lipids, one or more liposomes, one or more peptides, and one or more lipopeptides.

8. The method of claim 1, wherein said method results in an antibody response.

9. The method of claim 1, wherein said method induces an immune response.

10. The method of claim 9, wherein said immune response is a humoral immune response.

11. The method of claim 9, wherein said immune response is a T-cell mediated immune response.

12. The method of claim 9, wherein said immune response is a prophylactic immune response.

13. The method of claim 9, wherein said immune response is a therapeutic immune response.

14. The method of claim 1, wherein said organism is a mammal.

15. The method of claim 1, wherein said organism is a plant.

16. The method of claim 14, wherein said mammal is a human.

17. The method of claim 1, wherein said device for delivering is an electroporation device that delivers said formulation to said cell by pulse voltage.

18. The method of claim 1, wherein said delivering of said formulation comprises subjecting said cells to an electric field.

19. A kit comprising a container for providing a formulation comprising a nucleic acid molecule and a transfection facilitating agent, and either (i) a pulse voltage device for delivering said formulation to cells of an organism, wherein said pulse voltage device is capable of being combined with said container, or (ii) instructions explaining how to deliver said formulation with said pulse voltage device.

20. The kit of claim 19, wherein said nucleic acid molecule is DNA.

21. The kit of claim 19, wherein said nucleic acid molecule is a plasmid with a eukaryotic promoter which expresses a gene.

22. The kit of claim 21, wherein said gene is human growth hormone.

23. The kit of claim 19, wherein said nucleic acid molecule is RNA.

24. The kit of claim 19, wherein said transfection facilitating agent is a protective, interactive and non-condensing compound.

25. The kit of claim 19, wherein said transfection facilitating agent is selected from the group consisting of: one or more polyvinyl-pyrrolidones, one or more cationic lipids, one or more cationic lipids with neutral co-lipids, one or more liposomes, one or more peptides, and one or more lipopeptides.

26. The kit of claim 19, wherein said pulse voltage means for delivering is an electroporation device that injects said nucleic acid molecule by pulse voltage delivery into the cells of an organism.

27. The kit of claim 19, wherein said delivering of said formulation comprises subjecting said cells to an electric field.

28. A method for making a kit of claim 19 comprising the steps of combining a container for providing a formulation comprising a nucleic acid and a transfection facilitating agent with either (i) a pulse voltage device for delivering said formulation to the cells of an organism, wherein said pulse voltage device is capable of being combined with said container, or (ii) instructions explaining how to deliver said formulation with said pulse voltage device.

29. The method of claim 28, wherein said nucleic acid molecule is DNA.

30. The method of claim 28, wherein said nucleic acid molecule is a plasmid with a eukaryotic promoter which expresses a gene.

31. The method of claim 30, wherein said gene is for human growth hormone.

32. The method of claim 28, wherein said nucleic acid molecule is RNA.

33. The method of claim 28, wherein said transfection facilitating agent is a protective, interactive and non-condensing compound.

34. The method of claim 28, wherein said transfection facilitating agent is selected from the group consisting of: one or more polyvinyl-pyrrolidones, one or more cationic lipids, one or more cationic lipids with neutral co-lipids, one or more liposomes, one or more peptides, and one or more lipopeptides.

35. The method of claim 28, wherein said pulse voltage means for delivering is an electroporation device that injects said nucleic acid molecule by pulse voltage delivery to the cells of an organism.

36. A method of treating a mammal suffering from a disorder conventionally treated by administering human growth hormone, comprising the step of providing a formulation comprising a nucleic acid molecule encoding human growth hormone and a transfection facilitating agent to cells of said mammal by use of a device configured and arranged to cause pulse voltage delivery of said formulation to cells of said mammal.

37. The method of claim 36, wherein said mammal is a human.

38. The method of claim 36, wherein said transfection facilitating agent is a protective, interactive and non-condensing compound.

39. The method of claim 36, wherein said transfection facilitating agent is selected from the group consisting of: one or more polyvinyl-pyrrolidones, one or more cationic lipids, one or more cationic lipids with neutral co-lipids, one or more liposomes, one or more peptides, and one or more lipopeptides.

40. The method of claim 36, wherein said pulse voltage device for delivering is an electroporation device that injects said formulation by pulse voltage delivery to cells of a mammal.

41. A method of treating a mammal suffering from cancer, comprising the step of providing a formulation, said formulation comprising a nucleic acid molecule and a transfection facilitating agent to cells of said mammal by use of a device configured and arranged to pulse voltage delivery of formulation molecule to cells of said mammal, wherein said molecule encodes a cancer antigen.

42. The method of claim 41, wherein said mammal is a human.

43. The method of claim 41, wherein said cancer antigen is MAGE 1, and said cancer is melanoma.

44. The method of claim 41, wherein said transfection facilitating agent is a protective, interactive and non-condensing compound.

45. The method of claim 41, wherein said transfection facilitating agent is selected from the group consisting of: one or more polyvinyl-pyrrolidones, one or more cationic lipids, one or more cationic lipids with neutral co-lipids, one or more liposomes, one or more peptides, and one or more lipopeptides.

46. The method of claim 41, wherein said pulse voltage device for delivering is an electroporation device that injects said formulation by pulse voltage delivery to cells of a mammal.

47. A method of treating a mammal suffering from an infectious disease, comprising the step of providing a formulation, said formulation comprising a nucleic acid molecule and a transfection facilitating agent, to cells of said mammal by use of a device configured and arranged to cause pulse voltage delivery of said formulation to cells of said mammal, wherein said molecule encodes an antigen for said infectious disease.

48. The method of claim 47, wherein said mammal is a human.

49. The method of claim 47, wherein said infectious disease antigen is HBV core antigen, and said infectious disease is chronic hepatitis.

50. The method of claim 47, wherein said transfection facilitating agent is a protective, interactive and non-condensing compound.

51. The method of claim 47, wherein said transfection facilitating agent is selected from the group consisting of: one or more polyvinyl-pyrrolidones, one or more cationic lipids, one or more cationic lipids with neutral co-lipids, one or more liposomes, one or more peptides, and one or more lipopeptides.

52. The method of claim 47, wherein said device for delivering is an electroporation device that injects said nucleic acid molecule by pulse voltage delivery to cells of a mammal.

53. The cationic lipids of any of claim 25, 34, 39, 45, or 51, wherein at least one of said cationic lipids is DOTMA.

54. The neutral co-lipid of any of claim 25, 34, 39, 45, or 51, wherein said neutral co-lipid is cholesterol.

55. A method for delivering a nucleic acid molecule to a companion animal comprising the step of providing a formulation comprising said nucleic acid molecule and a transfection facilitating agent to the cells of said organism by use of a device configured and arranged to cause pulse voltage delivery of said formulation.

56. A method for delivering a nucleic acid molecule to a domestic animal comprising the step of providing a formulation comprising said nucleic acid molecule and a transfection facilitating agent to the cells of said organism by use of a device configured and arranged to cause pulse voltage delivery of said formulation.
